(19) United States
(12) Patent Application Publication
(10) Pub. No.: US 2021/0283168 A1
(43) Pub. Date: Sep. 16, 2021

(54) BETA GLUCAN AND CD40 AGONIST COMBINATION IMMUNOTHERAPY

(71) Applicants: HiberCell, Inc., New York, NY (US); THE TRUSTEES OF THE UNIVERSITY OF PENNSYLVANIA, Philadelphia, PA (US)

(72) Inventors: Nandita Bose, Plymouth, MN (US); Gregory Beatty, Philadelphia, PA (US)

(21) Appl. No.: 16/980,064

(22) PCT Filed: Mar. 13, 2019
(86) PCT No.: PCT/US2019/022062

§ 371 Date: Sep. 11, 2020

Related U.S. Application Data
(60) Provisional application No. 62/642,210, filed on Mar. 13, 2018.

(51) Int. Cl. A61K 31/716; C07K 16/28

(52) U.S. Cl. CPC A61K 31/716; C07K 2317/75; C07K 16/2878

(57) ABSTRACT

The present invention relates to the combination of β-glucan and a CD40 agonist for cancer immunotherapy. The combination therapy shows synergistic anti-tumor activity that is dependent on T cells. Surprisingly, the combination therapy is effective against poorly immunogenic tumors.

### CROSS-REFERENCE TO RELATED APPLICATION

This application claims priority to U.S. Provisional Patent Application Ser. No. 62/642,210 filed Mar. 13, 2018, which is incorporated herein by reference.

### SUMMARY

This disclosure provides, in one aspect, compositions of soluble β-glucan and a CD40 agonist and their use for cancer immunotherapy. The CD40 agonist may be an agonistic CD40 antibody and the soluble β-glucan may be derived from yeast.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIGS. 1A-1E. Tumor growth curves for treatment combinations including soluble β-glucan and FGK45.

FIGS. 2A-2E. Tumor growth curves for treatment combinations including soluble β-glucan and Gemcitabine chemotherapy.

FIGS. 3A-3B. Graphs comparing tumor growth between treatment groups.

FIG. 4. Graph comparing overall survival between treatment groups.

FIG. 5A. Graph comparing response between treatment groups.

FIG. 5B. Graph comparing overall survival between treatment groups.

FIGS. 5C-5E. Graphs comparing pharmacodynamic changes between treatment groups.

### DETAILED DESCRIPTION OF ILLUSTRATIVE EMBODIMENTS

Drugs designed to harness the immune system to treat cancer have shown promising results but, when used individually, many patients do not respond to these drugs or responses are transient. Thus, certain immunotherapeutic drugs may be combined to bridge the innate and adaptive arms of the immune system and drive enhanced cancer therapy. Specifically, the present invention is directed to compositions of β-glucan and CD40 agonists and their use in cancer immunotherapy.

β-glucans are polymers of glucose derived from a variety of microbiological and plant sources including, for example, yeast, bacteria, algae, seaweed, mushroom, oats, and barley. Of these, yeast β-glucans have been extensively evaluated for their immunomodulatory properties. Yeast β-glucans can be present as various forms such as, for example, intact yeast, zymosan, purified whole glucan particles, solubilized zymosan polysaccharide, or highly-purified soluble β-glucans of different molecular weights. Structurally, yeast β-glucans are composed of glucose monomers organized as a β-(1,3)-linked glucopyranose backbone with periodic β-(1,3) glucopyranose branches linked to the backbone via β-(1,6) glycosidic linkages. The different forms of yeast β-glucans can function differently from one another. The mechanism through which yeast β-glucans exert their immunomodulatory effects can be influenced by the structural differences between different forms of the β-glucans such as, for example, its particulate or soluble nature, tertiary conformation, length of the main chain, length of the side chain, and frequency of the side chains. The immune stimulating functions of yeast β-glucans are also dependent upon the receptors engaged in different cell types in different species, which again, can be dependent on the structural properties of the β-glucans.

In general, β-glucan immunotherapies can include administering to a subject any suitable form of β-glucan or any combination of two or more forms of β-glucan. Suitable β-glucans and the preparation of suitable β-glucans from their natural sources are described in, for example, U.S. Pat. No. 9,610,303. In some cases, the β-glucan may be derived from a yeast such as, for example, Saccharomyces cerevisiae. In certain cases, the β-glucan may be or be derived from β(1,6)-[poly-(1,3)-D-glucopyranosyl]-poly-β(1,3)-D-glucopyranose, also referred to herein as PGG (IMPRIME PGG, Biothera, Eagan, Minn.), a highly purified and well characterized form of soluble yeast-derived β-glucan. β-glucan is a PAMP (pathogen-associated molecular pattern) capable of triggering innate immune cell function leading to a cascade of immune activation and enhanced anti-tumor killing. Yeast soluble β-glucan has been known to enhance the direct killing of tumor cells by innate immune effector cells in the presence of tumor-targeting antibodies. However, yeast soluble β-glucan also enhances T cell-based cancer therapies through two mechanisms. Specifically, yeast soluble β-glucan triggers re-polarization of myeloid function to counteract the immune suppressive microenvironment established by a tumor. In addition, yeast soluble β-glucan activates the maturation of antigen presenting cells (i.e., macrophages and dendritic cells) enabling T cell expansion and activation as well as expression of IFN-γ. Unlike other PAMPs where systemic administration often leads to toxic side effects, Imprime PGG has been administered safely by intravenous infusion to more than 400 human subjects. Moreover, β-glucan-based immunotherapies can involve the use of, for example, a modified and/or derivatized β-glucan such as those described in International Patent Application No. PCT/US12/36795. In other cases, β-glucan immunotherapy can involve administering, for example, a particulate-soluble β-glucan or a particulate-soluble β-glucan preparation, each of which is described in, for example, U.S. Pat. No. 7,981,447.

Biomarker research demonstrated differences among subjects in the ability of their neutrophils and monocytes to bind yeast soluble β-glucan. Binding of yeast soluble β-glucan to these cells correlated with the subjects' immunomodulatory response to yeast soluble β-glucan. Moreover, yeast soluble β-glucan binding to neutrophils and monocytes involves the presence of a specific level of natural anti-β-glucan antibodies. Biomarker assay methods to quantitatively measure anti-β-glucan IgG and IgM antibodies (ABAs) in patient serum samples are described in International Published Application Nos. WO2013165591A1 and WO2015084732A1. Cutoff levels for the biomarker assay stratify subjects into biomarker positive and biomarker negative subgroups and these cutoff points correlate with binding, function, and clinical outcomes.

If a subject is identified as biomarker negative because the subject's level of ABAs falls below a selected, predetermined cutoff level, an antibody preparation capable of converting the subject from biomarker negative status to biomarker positive status can be administered to improve the subject's response to the immunotherapy. Thus, in some cases, the β-glucan and CD40 agonist combination immunotherapies may also include administration of an antibody preparation to improve the subject's response to the immunotherapy. Compositions and methods for improving a subject's response to immunotherapy are described in International Published Application No. WO2013165593A1.

In some cases, the antibody preparation can include serum from a biomarker positive subject. In some cases, the antibody preparation can include a monoclonal antibody or antibody fragment that specifically binds the β-glucan. In some cases, the antibody preparation can include intravenous immunoglobulin.

The CD40 molecule is a member of the TNF receptor superfamily and is expressed by multiple cell types including monocytes, macrophages, dendritic cells, B cells, platelets, fibroblasts, endothelial cells, smooth muscle cells and some malignant epithelial cells. The ligand for CD40, CD40 ligand (CD154), is expressed on a variety of cells including activated CD4 T cells, activated B cells, memory CD8 T cells, activated natural killer cells, granulocytes, endothelial cells, smooth muscle cells, and activated platelets. Activation of the CD40 pathway is a critical step in ‘licensing’ antigen presenting cells with the capacity to effectively present antigen and stimulate antigen-specific T cells. Ligation of CD40 on dendritic cells (DCs) enhanced the expression of costimulatory (e.g. CD80 and CD86) and major histocompatibility (MHC) molecules, induced the release of immunostimulatory cytokines and activated antigen presentation machinery. The importance of CD40 in tumor immunity was subsequently demonstrated in several studies where administration of an agonistic antibody directed against CD40 produced protective T cell immunity in murine models of cancer. This was the basis for the development of clinical grade CD40 agonists that are currently in clinic studies.

The CD40 agonist useful in the present invention may be a recombinant human CD40 ligand such as Avrend (Immunex Corp, Seattle, Wash.), CD40 ligand gene therapy such as an adenoviral vector serotype 5 carrying the human CD40L gene driven by a Rous sarcoma virus promoter (AdCD40L) (Vector Production Facility at the Center for Cell and Gene Therapy, Baylor College of Medicine, Houston, Tex.) or agonistic CD40 antibodies. Agonistic CD40 antibodies may be polyclonal antibodies, antibody fragments or monoclonal antibodies of various isotypes such as, for example, fully human IgG2, humanized rabbit IgG1, fully human IgG1, chimeric IgG1 and non-fucosylated humanized IgG1 isotypes.

Agonistic CD40 antibodies having an IgG1 Fc domain may be beneficial in some cases, because the IgG1 Fc domain enhances binding to Fcγ receptor II-B (FcγRIIB), which potentially increases CD40 agonist potency through crosslinking. Alternatively, agonistic CD40 antibodies may be chemically crosslinked for Fc-independent enhanced activity. In other cases, agonistic CD40 antibodies with an IgG2 Fc domain may mediate FcγR-independent agonistic activity through the unique hinge properties of the IgG2 isotype. Alternatively, in some cases, neither the Fc domain of the antibody nor Fc receptor crosslinking are required for CD40 stimulation by agonistic CD40 antibodies. The precise mechanism underlying this finding is unclear but could be explained by certain Fab regions binding to a specific epitope on human CD40 that produces potent signaling activity.

FcγRIIB is the only inhibitory Fc receptor and variations in the gene encoding this protein, or polymorphisms, have long been associated with susceptibility to autoimmune disease. These polymorphisms could have therapeutic implications related to the efficacy of IgG1-modified CD40 agonists such that various FcγRIIB polymorphisms would correlate with administration of specific CD40 antibodies. For example, subjects with FcγRIIB polymorphisms correlating with reduced efficacy of IgG1-modified CD40 agonists would only be administered IgG2 CD40 agonists or agonistic CD40 antibodies lacking the Fc domain.

In addition, in murine models, the enhanced potency of IgG1 CD40 agonists is associated with an increased risk of transient thrombocytopenia. Reducing the dose of the IgG1 CD40 agonist diminished the level of thrombocytopenia while still maintaining improved agonist activity compared to the IgG2 isotype. Thus, in some cases, a reduced dose or less frequent dosing of an IgG1 CD40 agonist may be preferred for the β-glucan and agonistic CD40 antibody combined immunotherapy.

Several agonistic CD40 monoclonal antibodies have been developed for clinical use that may be used in the present invention. Each antibody is distinct with unique properties defined by (i) binding affinity to CD40, (ii) isotype, (iii) dependence on crosslinking for activity and (iv) the ability to block CD40 ligand binding. These include:

(1) CP-870,893 (Pfizer and VLST) is an anti-CD40 IgG2 antibody with poor FcR binding that does not block CD40 ligand interaction with CD40; can mediate CD40 stimulation in the absence of cross-linking; and has a binding affinity (Kd) of 3.48×10⁻¹⁰ M.

(2) APX005 (Apexigen) is an IgG1 antibody recognizing CD40 that blocks CD40 ligand binding; shows enhanced activity in vitro with cross-linking; and has a Kd of 9.6×10⁻¹⁰ M.

(3) ADC-1013 (Alligator Bioscience) is an IgG1 antibody that recognizes CD40 with high binding affinity (Kd 1×10⁻¹¹ M) even under acidic conditions (pH 5.4) with activity dependent on FcR binding and crosslinking.

(4) Dacetuzumab (also called SGN-40 and formerly SGN-14) (Seattle Genetics) is a humanized IgG1 antibody recognizing CD40 with a Kd of 1×10⁻⁹ M. Dacetuzumab is a partial agonist that shows weak activity in stimulating B cell proliferation; displays potent anti-proliferative and pro-apoptotic properties against B cell lymphoma lines and enhances CD40 ligand binding to CD40.

(5) SEA-CD40 (Seattle Genetics) is a non-fucosylated humanized IgG1 anti-CD40 antibody derived from dacetuzumab (SGN-40) with a Kd of 1×10⁻⁹ M. SEA-CD40 shows improved agonist activity due to enhanced binding to FcgRIIIa.

(6) ChiLob 7/4 (University of Southamptom) is a chimeric IgG1 antibody with a Kd of 2×10⁻¹⁰ M that requires crosslinking for CD40 stimulation in antigen-presenting cells.

Poor responsiveness to immunotherapy that is seen in many patients may be due to the absence of an existing ongoing immune response against the cancer that is necessary for immunotherapy to be successful. As such, the present invention includes novel approaches, such as the use of immune agonists, capable of invoking potent antitumor immunity to broaden and enhance the therapeutic impact of cancer immunotherapy.

The efficacy of CD40 agonists has been reported to be dependent upon the presence of tumor antigen, which is necessary for CD40-activated antigen-presenting cells to induce antigen-specific T cell immunity. Subsequent studies were then focused on combining CD40 agonists with vaccines and chemotherapies to provoke an immunogenic form of tumor cell death. However, even in combination with chemotherapy, evidence for T cell-dependent antitumor activity was not observed and further studies identified immunosuppressive macrophages in the tumor microenvironment. These macrophages in addition to other immunosuppressive mechanisms likely explain the lack of T cell-dependent antitumor activity.

Soluble β-glucan binds to CD11b on cells of myeloid origin, namely neutrophils and monocytes, and increases the immunostimulatory functions of N1 neutrophils and M1 macrophages and decreases the immunosuppressive functions of myeloid-derived suppressor cells, N2 neutrophils and M2 macrophages. This modulation results in cross-talk between the various innate and adaptive cells in the tumor microenvironment, which redirects conditions toward immunostimulation. Soluble β-glucan directly affects M2 repolarization to the M1 phenotype and drives Th1 polarization, and soluble β-glucan-primed innate immune cells generate cytokines to indirectly affect CD4 and CD8 T cell proliferation, even in the presence of T-regulatory cells, and eventually drive Th1 polarization.

To this end, soluble β-glucan can be combined with CD40 agonists. For example, soluble β-glucan can be combined with agonistic CD40 antibodies in the treatment of cancers including, for example, melanoma, pancreatic cancer, multiple myeloma, non-Hodgkin's lymphoma, chronic lymphocytic leukemia, mesothelioma, advanced solid tumors, etc. Soluble β-glucan triggers re-polarization of myeloid function to counteract the immune suppressive microenvironment established by a tumor and activates the maturation of antigen-presenting cells leading to T cell expansion and activation and IFN-γ expression. These conditions are ideal for CD40 agonists to induce T cell-dependent antitumor immunity. The combination of a CD40 agonist and soluble β-glucan results in a synergistic effect that greatly enhances therapeutic outcomes even against poorly immunogenic tumors.

The invention includes, in part, co-administering a β-glucan with a CD40 agonist. As used herein, “co-administered” refers to two or more components of a combination administered so that the therapeutic or prophylactic effects of the combination can be greater than the therapeutic or prophylactic effects of either component administered alone. Two components may be co-administered simultaneously or sequentially. Simultaneously co-administered components may be provided in one or more pharmaceutical compositions. Sequential co-administration of two or more components is independent of timing and includes cases in which the components are administered so that both components are simultaneously bioavailable after both are administered. Regardless of whether the components are co-administered simultaneously or sequentially, the components may be co-administered at a single site or at different sites.

The β-glucan, the CD40 agonist, and/or the combination of both components may be formulated in a composition along with a “carrier.” As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutically active substances is well known in the art. Except insofar as any conventional media or agent is incompatible with the β-glucan or the CD40 agonist, its use in the therapeutic compositions is contemplated. Supplementary active ingredients also can be incorporated into the compositions.

By “pharmaceutically acceptable” is meant a material that is not biologically or otherwise undesirable, i.e., the material may be administered to an individual along with the β-glucan and/or the pharmaceutical agent without causing any undesirable biological effects or interacting in a deleterious manner with any of the other components of the pharmaceutical composition in which it is contained.

The β-glucan, the CD40 agonist, and/or the combination of both components may be formulated into a pharmaceutical composition. In some embodiments, the β-glucan and the CD40 agonist may be provided in a single formulation. In other embodiments, the β-glucan and the CD40 agonist may be provided in separate formulations. A pharmaceutical composition may be formulated in a variety of and/or a plurality forms adapted to one or more preferred routes of administration. Thus, a pharmaceutical composition can be administered via one or more known routes including, for example, oral, parenteral (e.g., intradermal, transcutaneous, subcutaneous, intramuscular, intravenous, intraperitoneal, intratumoral, etc.), or topical (e.g., intranasal, intrapulmonary, intramammary, intravaginal, intrauterine, intradermal, transcutaneous, rectally, etc.). A pharmaceutical composition, or a portion thereof, can be administered to a mucosal surface, such as by administration to, for example, the nasal or respiratory mucosa (e.g., by spray or aerosol). A pharmaceutical composition, or a portion thereof, also can be administered via a sustained or delayed release.

A formulation may be conveniently presented in unit dosage form and may be prepared by methods well known in the art of pharmacy. Methods of preparing a composition with a pharmaceutically acceptable carrier include the step of bringing the β-glucan and/or the Cd40 agonist into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

The β-glucan, the CD40 agonist, and/or the combination of both components may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

In some embodiments, the invention can include administering sufficient β-glucan to provide a dose of, for example, from about 100 ng/kg to about 50 mg/kg to the subject, although in some embodiments the β-glucan may be administered in a dose outside this range. The dose may be dependent on a subject's ABA levels. In some embodiments, the β-glucan dose range is about 0.5 mg/kg to about 6 mg/kg including 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg and 6.0 mg/kg. The β-glucan may only be dosed once or time intervals between dosing may be, for example, every 1, 2, 3, 4, 5, 6, 7, 8, 9, 10, 11 or 12 weeks during a course of treatment or every 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83 or 84 days. The dosing schedule will vary depending on, among other things, the cancer being treated and the subject's ABA level. Soluble β-glucan dosing is described in International Patent Application No. PCT/US18/19412.

Alternatively, the dose may be calculated using actual body weight obtained just prior to the beginning of a treatment course. For the dosages calculated in this way, body surface area (m²) is calculated prior to the beginning of the treatment course using the Dubois method: m²=(wt kg^{0.425}×height cm^{0.725})×0.007184. In some embodiments, therefore, the method can include administering sufficient β-glucan to provide a dose of, for example, from about 0.01 mg/m² to about 10 mg/m².

The invention includes administering sufficient CD40 agonist. The dose of CD40 agonist may be from 0.01 mg/kg to 20 mg/kg, including 0.01 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.5 mg/kg, 10.0 mg/kg, 10.5 mg/kg, 11.0 mg/kg, 11.5 mg/kg, 12.0 mg/kg, 12.5 mg/kg, 13.0 mg/kg, 13.5 mg/kg, 14.0 mg/kg, 14.5 mg/kg, 15.0 mg/kg, 15.5 mg/kg, 16.0 mg/kg, 16.5 mg/kg, 17.0 mg/kg, 17.5 mg/kg, 18.0 mg/kg, 18.5 mg/kg, 19.0 mg/kg, 19.5 mg/kg and 20.0 mg/kg. The CD40 agonist may only be dosed once or time intervals between dosing may be, for example, every 1, 2, 3, 4, 5, 6, 7, 8, 9, 10, 11 or 12 weeks during a course of treatment or every 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83 or 84 days.

Alternatively, the dose of either β-glucan or CD40 agonist may be calculated using actual body weight obtained just prior to the beginning of a treatment course. For the dosages calculated in this way, body surface area (m²) is calculated prior to the beginning of the treatment course using the Dubois method: m²=(wt kg^{0.425}×height cm^{0.725})×0.007184. In some embodiments, therefore, the method can include administering sufficient β-glucan and/or CD40 agonist to provide a dose of, for example, from about 0.01 mg/m² to about 10 mg/m².

The present invention is illustrated by the following examples. It is to be understood that the particular examples, materials, amounts, and procedures are to be interpreted broadly in accordance with the scope and spirit of the invention as set forth herein.

### Example 1

Soluble β-glucan in combination with a CD40 agonist enhances tumor regression: The role for soluble β-glucan in inducing potent anti-tumor immunity against pancreatic ductal adenocarcinoma (PDAC), a poorly immunogenic cancer that is marked by T cell exclusion and a robust inflammatory immune reaction, was evaluated. In this study, C57BL/6 mice were implanted subcutaneously with a KPC-derived PDAC cell line (7940B, P9). Mice were monitored 2-3 times per week for tumor growth by calipers. On day −2, mice received gemcitabine (120 mg/kg) or PBS. On day 0, mice were treated with or without FGK45 (0.1 mg), IMPRIME PGG (1.2 mg), or both. FGK45 is a rat anti-mouse monoclonal agonistic antibody to CD40 and was administered by intraperitoneal injection. IMPRIME PGG was administered by intravenous injection.

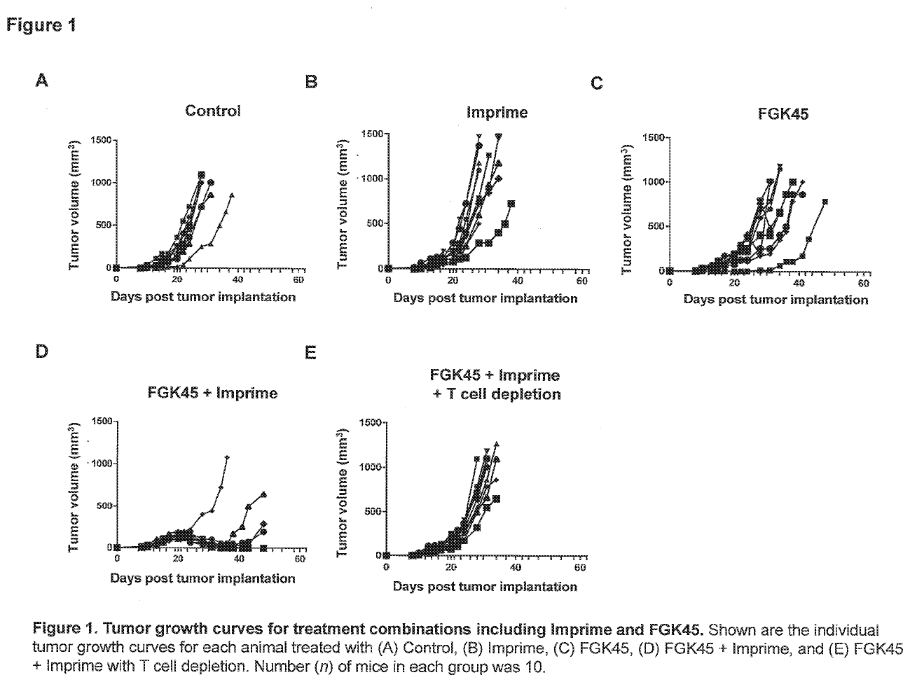

Mice were followed for tumor growth and overall survival (i.e. until tumors reached >1000 mm³ or animals demonstrated tumor-related discomfort/distress). The impact of IMPRIME PGG treatment alone on tumor growth versus the control is shown in FIGS. 1A and 1B. Here, treatment with a single dose of IMPRIME PGG produced a delay in tumor outgrowth in 1 of 10 mice. In a previous experiment, IMPRIME PGG produced a delay in tumor outgrowth in 3 of 9 mice (data not shown). These experiments indicate that IMPRIME PGG has limited activity as monotherapy in this tumor model. Similar results, shown in FIG. 1C, were seen with FGK45 treatment alone where delayed tumor growth was seen in 4 of 10 mice. A previous experiment showed delayed tumor growth in 5 of 9 mice (data not shown).

In contrast, combining IMPRIME PGG and FGK45 produced tumor regressions in 9 of 10 mice (FIG. 1D). Complete tumor regressions were seen in 6 of 10 mice without subsequent recurrence.

### Example 2

Soluble β-glucan in combination with an agonistic CD40 antibody invokes T cell-dependent antitumor activity against a poorly immunogenic tumor: On days −3, 0, 4, 7 and 11, mice from Example 1 were treated with or without GK1.5 (anti-CD4) and 2.43 (anti-CD8) depleting antibodies (0.2 mg/dose). 2.43 and GK1.5 are rat anti-mouse monoclonal antibodies that deplete CD8a and CD4 expressing T cells, respectively. Both antibodies were administered by intraperitoneal injection.

As shown in FIG. 1E, the synergistic therapeutic activity using the combination of IMPRIME PGG and FGK45 was ablated when CD4 and CD8 T cells were depleted. Thus, this data shows that combining IMPRIME PGG and FGK45 produces remarkable T cell-dependent anti-tumor activity in a poorly immunogenic model of murine pancreatic ductal adenocarcinoma.

### Example 3

Soluble β-glucan in combination with chemotherapy enhances tumor regression: C57BL/6 mice were implanted subcutaneously with a KPC-derived PDAC cell line (7940B, P9). Mice were monitored 2-3 times per week for tumor growth by calipers. On day −2, mice received gemcitabine (120 mg/kg) or PBS.

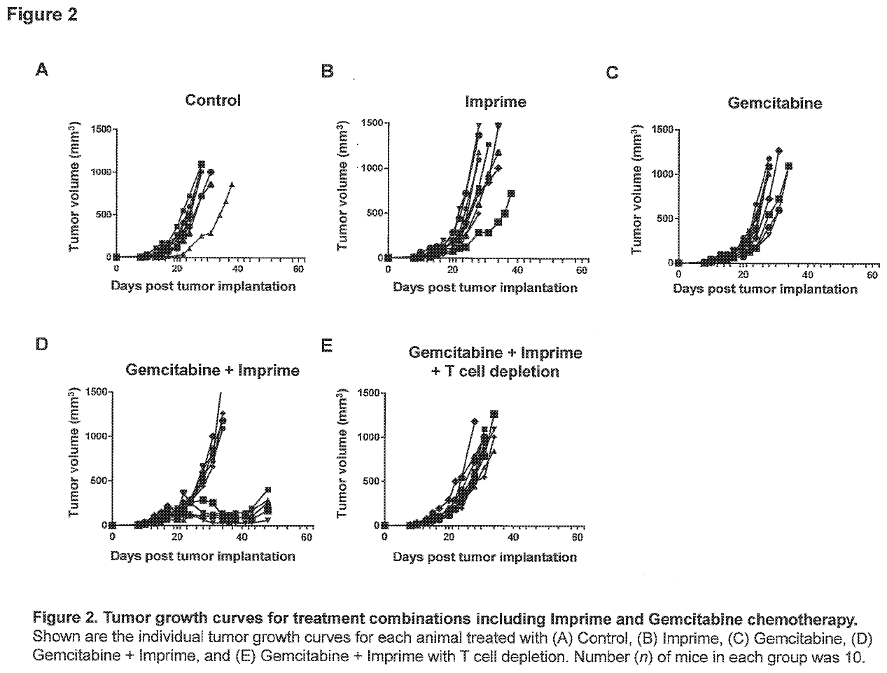

Again, IMPRIME PGG alone demonstrated minimal activity (FIG. 2A, B). Similarly, as shown in FIG. 2C, gemcitabine chemotherapy produced no significant impact on tumor growth. In contrast, treatment with gemcitabine chemotherapy administered 48 hours prior to IMPRIME PGG produced significant anti-tumor activity with delayed tumor outgrowth seen in 5 of 10 mice (FIG. 2D). In a previous experiment, this treatment combination produced anti-tumor activity in 6 of 9 mice.

### Example 4

Soluble β-glucan in combination with chemotherapy invokes T cell-dependent antitumor activity against a poorly immunogenic tumor: On days −3, 0, 4, 7 and 11, mice from Example 3 were treated with or without GK1.5 (anti-CD4) and 2.43 (anti-CD8) depleting antibodies (0.2 mg/dose). The synergistic therapeutic effect using the combination of IMPRIME PGG and gemcitabine is dependent on CD4 and CD8 T cells as seen when T cells are depleted prior to treatment (FIG. 2E). Thus, combining chemotherapy with IMPRIME PGG can produce significant T cell dependent anti-tumor activity in a poorly immunogenic model of murine pancreatic ductal adenocarcinoma.

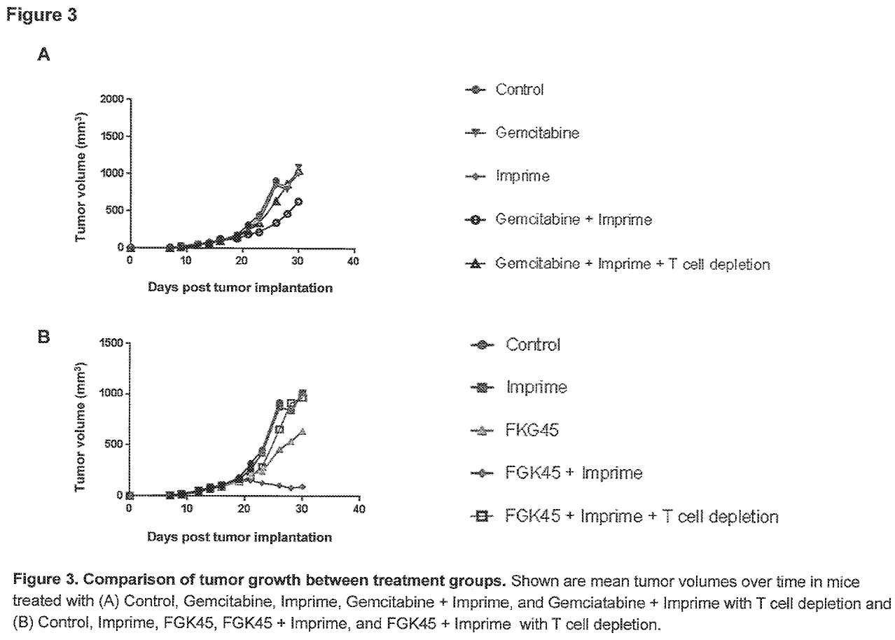

FIGS. 3A and 3B show the mean tumor growth curves for each gemcitabine and FGK45 treatment group, respectively. Statistical significance for tumor growth comparisons at day 26 was determined using two-way Anova with Tukey's multiple comparisons testing. Statistics between treatment groups are shown in Table 1.

TABLE 1

| Treatment Compared | P Value |
|---|---|
| Ctrl vs IMPRIME | 0.9944 |
| Ctrl vs FGK45 | <0.0001 |
| Ctrl vs Gemcitabine | 0.9750 |
| Ctrl vs FGK45 + IMPRIME | <0.0001 |
| Ctrl vs Gemcitabine + IMPRIME | <0.0001 |
| IMPRIME vs FGK45 | <0.0001 |
| IMPRIME vs Gemcitabine | >0.9999 |
| IMPRIME vs FGK45 + IMPRIME | <0.0001 |
| IMPRIME vs Gemcitabine + IMPRIME | <0.0001 |
| FGK45 vs Gemcitabine | 0.0001 |
| FGK45 vs FGK45 + IMPRIME | 0.0008 |
| FGK45 vs Gemcitabine + IMPRIME | 0.7767 |
| Gemcitabine vs FGK45 + IMPRIME | <0.0001 |
| Gemcitabine vs Gemcitabine + IMPRIME | <0.0001 |
| FGK45 + IMPRIME vs Gemcitabine + IMPRIME | 0.0601 |

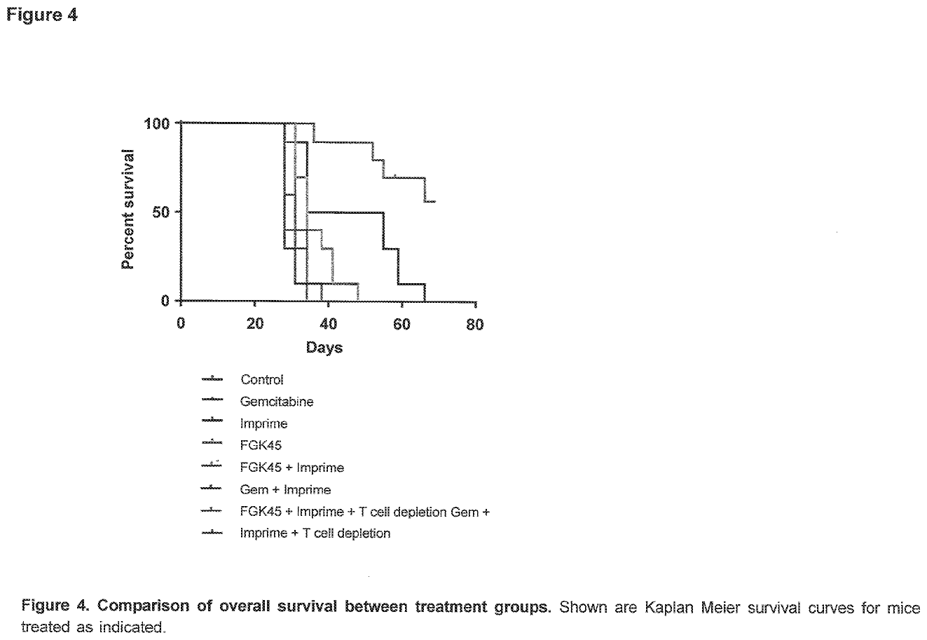

FIG. 4 shows the impact of each treatment on overall survival. Statistics for survival comparisons between treatment groups are shown in Table 2. Statistical significance was determined using Log Rank test.

TABLE 2

| Treatment Compared | P Value |
|---|---|
| Ctrl vs IMPRIME | 0.2794 |
| Ctrl vs FGK45 | 0.0024 |
| Ctrl vs Gemcitabine | 0.7345 |
| Ctrl vs FGK45 + IMPRIME | <0.0001 |
| Ctrl vs Gemcitabine + IMPRIME | 0.0002 |
| IMPRIME vs FGK45 | 0.0291 |
| IMPRIME vs Gemcitabine | 0.3989 |
| IMPRIME vs FGK45 + IMPRIME | <0.0001 |
| IMPRIME vs Gemcitabine + IMPRIME | 0.0029 |
| FGK45 vs Gemcitabine | 0.0074 |
| FGK45 vs FGK45 + IMPRIME | <0.0001 |
| FGK45 vs Gemcitabine + IMPRIME | 0.0284 |
| Gemcitabine vs FGK45 + IMPRIME | <0.0001 |
| Gemcitabine vs Gemcitabine + IMPRIME | 0.0012 |
| FGK45 + IMPRIME vs Gemcitabine + IMPRIME | 0.0048 |
| FGK45 + IMPRIME vs FGK45 + IMPRIME + | <0.0001 |
| T cell depletion | |
| Gemcitabine + IMPRIME vs Gemcitabine + | 0.0036 |
| IMPRIME + T cell depletion | |

Statistically significant differences were seen between the following treatment groups: (i) control vs gemcitabine +IMPRIME (p=0.0002), (ii) gemcitabine vs gemcitabine + IMPRIME (p=0.0012), (iii) IMPRIME vs gemcitabine + IMPRIME (p=0.0029), (iv) control vs FGK45+ IMPRIME (p<0.0001), (v) FGK45 vs FGK45+ IMPRIME (p<0.0001), (vi) IMPRIME vs FGK45+ IMPRIME (p<0.0001), and (vii) gemcitabine + IMPRIME vs FGK45+ IMPRIME (p =0.0048). Together, these data show remarkable therapeutic activity when either gemcitabine or FGK45 is combined with IMPRIME PGG, and that the therapeutic activity of both treatment combinations is dependent on T cells. Overall, a greater response rate (90% versus 50%) and improved overall survival (60% vs 0%) was seen when IMPRIME PGG was combined with FGK45 compared with chemotherapy, respectively.

Combining soluble β-glucan with a CD40 agonist is more efficacious than either treatment alone and produces complete and durable tumor regressions in 60% of mice. In addition, the anti-tumor effect of soluble β-glucan plus a CD40 agonist is dependent on CD4 and CD8 T cells.

### Example 5

Phagocytic myeloid cells are required for durability of the anti-tumor response of soluble β-glucan in combination with an agonistic CD40 antibody: The role of distinct myeloid cell subsets for anti-tumor activity induced with IMPRIME PGG and a CD40 agonist. Myeloid-targeting compounds were administered to deplete extratumoral macrophages (i.e. clodronate liposomes), to deplete Ly6C⁺ peripheral blood monocytes (i.e. anti-Ly6C antibodies), to deplete tumor-associated macrophages/extratumoral macrophages (i.e. colony-stimulating factor 1 receptor (CSF1R) inhibitor), and to block CD70 expressed by antigen-presenting cells including macrophages and dendritic cells (anti-CD70 antibodies).

C57BL/6 mice (n=10/group) were implanted subcutaneously with a KPC-derived PDAC cell line. Mice were monitored 2-3 times per week for tumor growth by calipers. When tumors reach approximately 4-5 mm in diameter, mice received the following treatments:

Group 1: Control

Group 2: FGK45+ IMPRIME PGG

Group 3: FGK45+ IMPRIME PGG+ clodronate liposomes (Gastroenterology. 2015 July; 149(1): 201-210.)

Group 4: FGK45+ IMPRIME PGG+ anti-Ly6C (Cancer Discov. 2016 April; 6(4): 400-413.)

Group 5: FGK45+ IMPRIME PGG+ CSF1R inhibitor

Group 6: FGK45+ IMPRIME PGG+CD70 blocking antibody (Nat Commun. 2012 Jul. 10; 3:948.)

The compounds were administered using the following dosing schema:

1. FGK45 was administered on day 0 at 0.1 mg by intraperitoneal (i.p.) injection

2. IMPRIME PGG was administered on day 0 at 1.2 mg intravenously (i.v.).

3. Clodronate encapsulated liposomes were administered i.p. at 10 mg/kg on days −2, 2, 5, and 9.

4. Anti-Ly6C antibodies (Monts1) were administered i.p. at 0.5 mg/dose on days −1, 0, 1, 5, 9.

5. CSF1R inhibitor (GW2580) was administered daily by oral gavage at 160 mg/kg on days −2 to 2 and days 5 to 9.

6. Anti-CD70 antibodies (clone FR70) was administered i.p. at 0.25 mg/dose on days −1, 0, 1, 5.

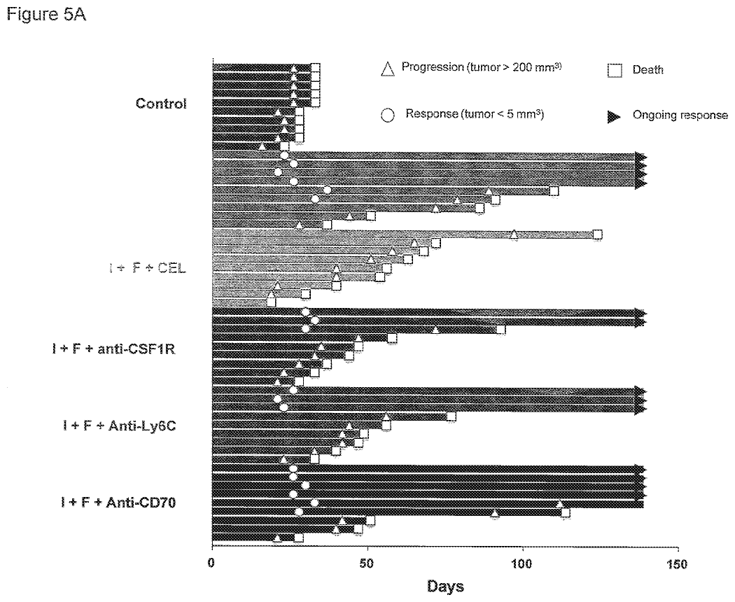

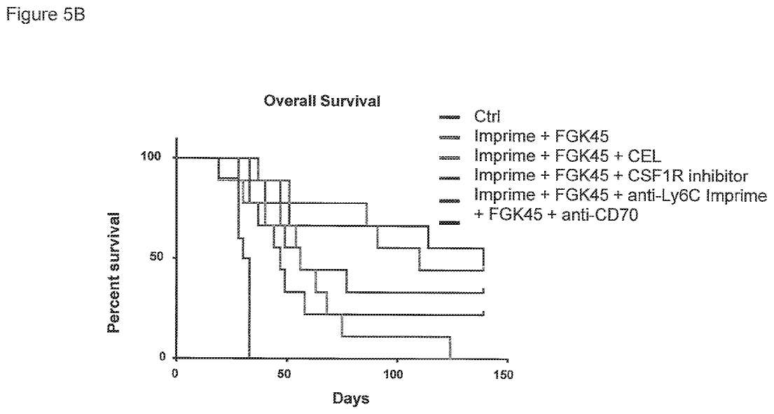

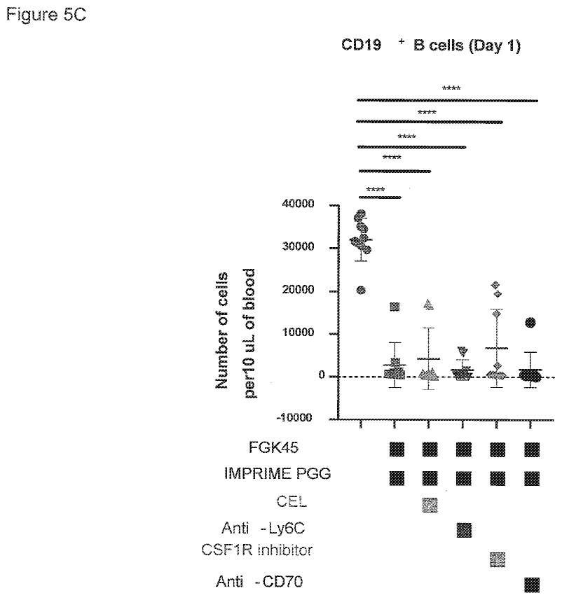

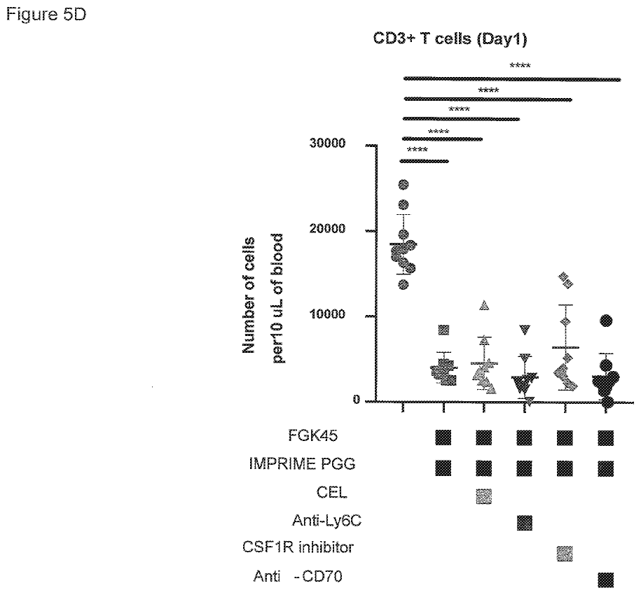

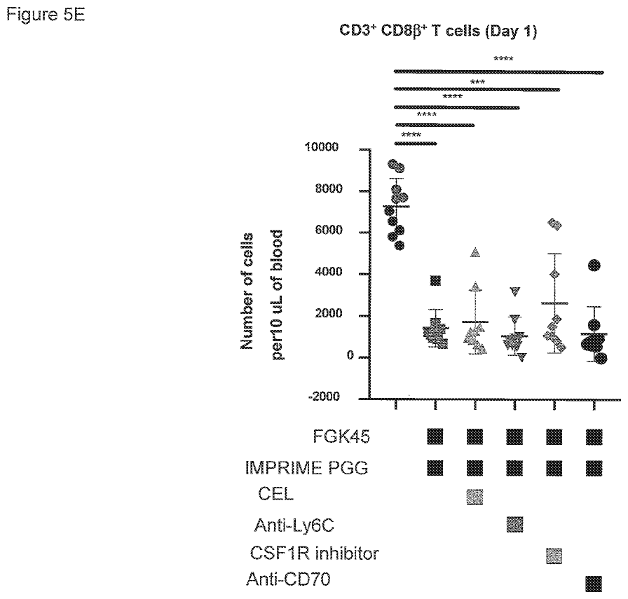

FIGS. 5A and 5B show the response to treatment and survival data for each group. The data indicate that phagocytic myeloid cells are required for response durability, because depletion of macrophages by clodronate encapsulated liposomes inhibited the combination therapy.

In addition, peripheral blood was collected on each mouse by tail vein bleed on day −2, 0 and 1 of treatment and analyzed by flow cytometry for the presence of CD19⁺ B cells, CD3⁺ T cells and CD3⁺ CD8β⁺ T cells. The results are shown in FIGS. 5C, 5D and 5E, and indicate that these cell types leave the peripheral blood circulation.

The synergistic effect that is seen with the combination CD40 agonist/soluble β-glucan therapy is unexpected and remarkable for at least three reasons. First, no chemotherapy is required. As discussed above, the efficacy of CD40 agonists has been reported to be dependent upon the presence of tumor antigen, which is necessary for CD40-activated antigen-presenting cells to induce antigen-specific T cell immunity. Example 2 shows that the combination therapy shows remarkable activity against a poorly immunogenic tumor without chemotherapy. Example 5 shows the requirement of phagocytic myeloid cells suggesting soluble β-glucan alters the phagocytic capacity of tumor-infiltrating myeloid cells and in doing so, improves their capture of tumor antigens. Second, not only is the activity observed with the combination CD40 agonist/soluble β-glucan therapy remarkable without chemotherapy, but it exceeds that seen using chemotherapy followed by a CD40 agonist. Third, it is unexpected and remarkable that the combination of two myeloid targeting agents, a CD40 agonist and soluble β-glucan, resulted in synergistic activity. Example 1 shows that monotherapy with either agent showed limited activity, but the combination CD40 agonist/soluble β-glucan therapy showed significant, more than additive, activity.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method of treating a subject having cancer, the method comprising administering soluble β-glucan and a CD40 agonist.

2. The method according to claim 1, wherein the cancer is a poorly immunogenic cancer.

3. The method according to claim 1, wherein the cancer is melanoma, pancreatic cancer, multiple myeloma, non-Hodgkin's lymphoma, chronic lymphocytic leukemia, mesothelioma or advanced solid tumors.

4. The method according to claim 1, wherein the soluble β-glucan and the CD40 agonist are in a single formulation.

5. The method according to claim 1, wherein the soluble β-glucan and the CD40 agonist are in separate formulations.

6. The method according to claim 1, wherein the soluble β-glucan is derived from yeast.

7. The method according to claim 6, wherein the yeast is Saccaromyces cerevisiae.

8. The method according to claim 1, wherein the soluble β-glucan is β(1,6)-[poly-(1,3)-D-glucopyranosyl]-poly-β(1,3)-D-glucopyranose.

9. The method according to claim 1, wherein the CD40 agonist is an agonistic CD40 antibody.

10. The method according to claim 9, wherein the agonistic CD40 antibody is a non-complement-activating antibody.

11. The method according to claim 9, wherein the agonistic CD40 antibody is an IgG1 or IgG2 antibody.

12. The method according to claim 9, wherein the agonistic CD40 antibody is CP-870,893, APX005, ADC-1013, Dacetuzumab, SEA-CD40 or ChiLob 7/4.

13. The method according to claim 1, wherein the method further comprises administering an anti-β-glucan antibody component.

14. A method of stimulating a subject's immune system against cancer cells, the method comprising administering soluble β-glucan and an agonistic CD40 antibody.

15. The method according to claim 14, wherein administration of the soluble β-glucan and an agonistic CD40 antibody results in a synergistic effect.

16. The method of claim 15 wherein the cancer cells are poorly immunogenic.

17. A composition for cancer immunotherapy comprising:
soluble β-glucan; and
an agonistic CD40 antibody.

18. The composition of claim 17 wherein the soluble β-glucan is β(1,6)-[poly-(1,3)-D-glucopyranosyl]-poly-β(1,3)-D-glucopyranose.

19. The composition of claim 17 wherein the agonistic CD40 antibody is an IgG1 or IgG2 antibody.

20. The composition of claim 17 wherein the soluble β-glucan and agonistic CD40 antibody have a synergistic effect against poorly immunogenic cancers.
